(19) United States
(12) Patent Application Publication
(10) Pub. No.: US 2019/0046595 A1
(43) Pub. Date: Feb. 14, 2019

(54) METHODS OF USING SMECTITE COMPOSITIONS FOR TREATING CLOSTRIDIUM DIFFICILE ASSOCIATED DISEASES AND SYMPTOMS

(71) Applicant: Platinum Performance, Inc., Buellton, CA (US)

(72) Inventors: Mark HERTHEL, Buellton, CA (US); Erin KELLEY, Buellton, CA (US); Tara HEMBROOKE, Buellton, CA (US); Chase MILLHOLLEN, Buellton, CA (US)

(21) Appl. No.: 16/163,222

(22) Filed: Oct. 17, 2018

Related U.S. Application Data
(63) Continuation of application No. 15/453,834, filed on Mar. 8, 2017, now abandoned.
Application No. 15/453,834 is a continuation of application No. 14/328,551, filed on Jul. 10, 2014, now abandoned.
(60) Provisional application No. 61/844,803, filed on Jul. 10, 2013.

(51) Int. Cl. A61K 36/064; A61K 38/05; A61K 35/742; A61K 35/745; A61K 35/747; A23L 33/16; A23L 33/135; A61K 31/702; A61K 31/198; A61K 33/06

(52) U.S. Cl. CPC A61K 36/064; A23V 2002/00; A61K 38/05; A61K 35/742; A61K 35/745; A61K 35/747; A23V 2250/15; A23L 33/135; A61K 31/702; A61K 31/198; A61K 33/06; A61K 2300/00; A23V 2200/32; A23L 33/16

(57) ABSTRACT

Provided herein are compositions useful for the treatment or prevention of C. difficile-associated disease or C. difficile-associated diarrhea in a subject in need thereof, and delivery systems and package systems comprising the same. Also provided are methods using the compositions, delivery systems, package systems and methods of making the same.

### CROSS-REFERENCE TO RELATED APPLICATIONS

This application claims the benefit of U.S. Provisional Application No. 61/844,803, filed Jul. 10, 2013, which is incorporated by reference in its entirety.

### FIELD

Provided herein are compositions useful for the treatment or prevention of Clostridium difficile associated disease or Clostridium difficile-associated diarrhea in a subject in need thereof. In certain aspects, the compositions comprise an effective amount of smectite and, optionally, an effective amount of at least one second agent (or at least one further agent) for the treatment or prevention of Clostridium difficile associated disease in a subject in need thereof. Also provided are methods using the compositions, delivery systems, package systems and methods of making the same.

### BACKGROUND

Antibiotics are among the most prescribed medications in the world, and they are commonly administered to hospitalized and non-hospitalized patients. While antibiotics can be effective to treat or prevent microbial infection, the same antibiotics can also disrupt the healthy flora of the gastrointestinal tract. Suppression of normal flora can permit Clostridium difficile (C. difficile) to colonize the gastrointestinal tract. C. difficile infection can cause disease (C. difficile-associated disease, or CDAD), for example C. difficile-associated diarrhea. The diarrhea can progress to colitis, dehydration, electrolyte disturbance, bowel perforation, megacolon, and possibly death. Gao et al., 2010, Am. J. Gastroenterol. 105:1636-1641.

In the United States alone, C. difficile is connected to 337,000 infections and 14,000 deaths each year according to the Centers for Disease Control and Prevention. Older adults who take antibiotics and also get medical care are currently most at risk for infection, and the incidence of infection in children is growing. Khanna et al., 2013, Clinical Infectious Diseases 56 (10): 1401-1406. C. difficile infection is now recognized outside the hospital in community-acquired diarrhea. Rodrigues et al., 2013, J. Infect. Diseases 207:1505-1515. The increasing use of acid-suppressing drugs is also attributed to the increase in C. difficile infections. Howell et al., 2010, Arch Int Med 170:784-90.

C. difficile is a spore-forming, gram-positive anaerobic bacterium that produces toxin A and toxin B. These toxins can cause the symptoms associated with C. difficile infection. The first step in C. difficile infection is for a person to become colonized with the organism. This can occur when the person comes into contact and ingests the organism (typically the spore form). Next, the organism establishes residence in the colon, which is often due to a reduction in the normal gastrointestinal bacteria (e.g., via antibiotic use). Next, the spore germinates into the vegetative form and produces toxins. These toxins attack the colonic epithelial cells, producing diarrhea, colonic pseudomembranes, toxic megacolon, and sometimes death. Adapted from Timothy Wiemken, “A case study for cleaning, disinfection, and process compliance: reducing transmission of C. difficile in the healthcare environment,” The APUA Newsletter, vol. 31, No. 1, pp. 10-12 (25 Apr. 2013).

Treatments for infection include discontinuation of antibiotic therapy, dietary changes, bed rest, and intravenous fluid. Gao et al. Some cases require new antibiotic therapy and/or fecal transplant. See id. Unfortunately, a significant number of patients treated for CDAD relapse within two months, and over half of patients with two or more previous episodes relapse again. See id. Additionally, standard treatment with metronidazole or vancomycin is a concern because of the potential for antibiotic-resistant bacteria as a result of this therapy.

New compositions and methods are needed for the treatment and prevention of C. difficile associated disease in subjects in need thereof.

### SUMMARY

Provided herein are compositions useful, for example, for the treatment and prevention of C. difficile-associated diseases and symptoms, including C. difficile-associated diarrhea. Also provided herein are methods of using the compositions, package systems comprising the compositions, delivery systems and methods of making the compositions.

In one aspect, provided herein is a composition comprising smectite. The composition can be, for example, a medical food, a nutritional supplement or a nutraceutical, or a component thereof. The smectite can be any smectite known to those of skill in the art. In certain embodiments, the smectite is di-tri-octahedral smectite. The composition can comprise any amount of smectite deemed suitable by those of skill in the art. In certain embodiments, the composition comprises a sufficient amount of smectite to treat or prevent C. difficile associated disease in a subject in need thereof.

In certain embodiments, the composition comprises smectite as the sole active ingredient. In such embodiments, the composition can comprise other ingredients such as binders, fillers, etc. In certain embodiments, the composition comprises one or more second agents (also referred to herein as “one or more further agents” or “at least one further agent”) effective to treat or prevent C. difficile associated disease in a subject in need thereof. The second agent can be any agent known to those of skill in the art to be effective for the treatment or prevention of C. difficile associated disease. In certain embodiments, the composition can further comprise one or more pharmaceuticals deemed suitable by the practitioner of skill.

While not intending to be bound by any particular theory of operation, certain embodiments described herein are based on the observation that smectite can effectively bind C. difficile toxin in the gut to treat and/or prevent symptoms of C. difficile associated disease in a subject in need thereof.

In another aspect, provided herein are methods of treating or preventing C. difficile associated disease in a subject in need thereof. The methods generally comprise administering a composition provided herein in an amount effective to treat or prevent the disease, or a symptom thereof. In certain embodiments, provided herein are methods of treating or preventing any symptom of C. difficile-associated disease in a subject in need thereof. In certain embodiments, provided herein are methods of treating or preventing C. difficile-associated diarrhea in a subject in need thereof. The methods generally comprise administering a composition provided herein in an amount effective to treat or prevent the disease, or a symptom thereof.

In certain embodiments, methods for the specific dietary management of a C. difficile associated disease, or a symptom thereof, are provided comprising the step of administering to a human subject in need thereof a composition provided herein. In certain embodiments, the subject has limited or impaired capacity to ingest, digest, absorb, or metabolize ordinary foodstuffs. In certain embodiments, the subject requires the composition as a major treatment modality. In certain embodiments, the administering is under medical supervision.

In another aspect, provided herein is a delivery system for delivering a composition provided herein. In certain embodiments, the delivery system comprises a composition provided herein and one or more components capable of delivering the composition to a host in need thereof.

In another aspect, provided herein is a package system for the compositions provided herein. In certain embodiments, the package system comprises a package that contains a composition provided herein. A package can comprise a single dose of the composition, or multiple doses of the composition. The package system can comprise a single package, or the package system can comprise multiple packages. The package system can further comprise a delivery system for delivering the compositions.

As described herein, the compositions, methods, delivery systems and/or package systems are useful for administering to subjects. In particular embodiments, they are useful for administering to subjects in need of treatment or prevention of C. difficile associated disease and/or a symptom associated with C. difficile associated disease.

### DESCRIPTION OF EXEMPLARY EMBODIMENTS

Provided herein are compositions useful for treating C. difficile-associated disease or C. difficile-associated diarrhea in a subject in need thereof, methods of their use and methods of making the same. Further provided are package systems and delivery systems useful for such methods.

### Definitions

When referring to the compositions provided herein, the following terms have the following meanings unless indicated otherwise. Unless defined otherwise, all technical and scientific terms used herein have the same meaning as is commonly understood by one of ordinary skill in the art. In the event that there is a plurality of definitions for a term herein, those in this section prevail unless stated otherwise.

As used herein, the term “smectite” refers to any clay mineral of the smectite group recognized by those of skill in the art. The term includes dioctahedral smectites such as montmorillonite and nontronite, dioctahedral smectites such as saponite, and di-tri-octahedral smectite. The smectite can be in any form or purity. In certain embodiments, the smectite is in the form of bentonite. Preferably, the amount of smectite is measured as the amount of pure smectite.

As used herein, the term “medical food” refers to a food which is formulated to be consumed or administered enterally under the supervision of a physician and which is intended for the specific dietary management of a disease or condition for which distinctive nutritional requirements, based on recognized scientific principles, are established by medical evaluation. Medical foods are those foods defined in section 5(b) of the U.S. Orphan Drug Act (21 U.S.C. § 360ee (b) (3)). In some embodiments, “medical foods” are specially formulated and processed products, meet distinctive nutritional requirements of a disease or condition, are used under medical supervision, and/or are intended for the specific dietary management of a disease or condition. In certain embodiments, “medical foods” are foods that are specially formulated and processed for a patient who is seriously ill or who requires the “medical food” as a major treatment modality. In certain embodiments, a “medical food” is provided for the dietary management of a patient who has limited or impaired capacity to ingest, digest, absorb, or metabolize ordinary foodstuffs, for example because of a therapeutic or chronic medical need. In certain embodiments, a “medical food” provides nutritional support specifically modified for the management of unique nutrient needs that result from a specific disease or condition, as determined by medical evaluation. In certain embodiments, a “medical food” is provided for use under medical supervision. In certain embodiments, a “medical food” is provided for a patient receiving active and on-going medical supervision.

As used herein, the term “nutritional supplement” refers to a composition intended to supplement the diet of an animal. In certain embodiments, the animal is a human. In certain embodiments, the “nutritional supplement” is selected from the group consisting of vitamins, minerals, herbs or other botanicals, amino acids, enzymes, organ tissues, glandulars, metabolites, extracts, concentrates, and combinations thereof. In certain embodiments, the “nutritional supplement” is a dietary supplement which helps to support and/or maintain a healthy digestive tract.

As used herein, the term “nutraceutical” refers to a product isolated or purified from food that is generally sold in medicinal forms not usually associated with food. In certain embodiments, a nutraceutical is demonstrated to have a physiological benefit or provide protection against chronic disease.

As used herein, EC₅₀ refers to a dosage, concentration or amount of a particular test agent that elicits a dose-dependent response at 50% of maximal expression of a particular response that is induced, provoked or potentiated by the particular test agent.

As used herein, the IC₅₀ refers to an amount, concentration or dosage of a particular test agent that achieves a 50% inhibition of a maximal response in an assay that measures such response.

As used herein, the terms “subject” and “patient” are used interchangeably. The terms “subject” and “subjects” refer to an animal, such as a mammal including a non-primate (e.g., a cow, pig, horse, cat, dog, rat, and mouse) and a primate (e.g., a monkey such as a cynomolgous monkey, a chimpanzee and a human), and for example, a human. In certain embodiments, the subject is a human.

“Treating” or “treatment” of any disease or disorder refers, in certain embodiments, to ameliorating a disease or disorder that exists in a subject. In another embodiment, “treating” or “treatment” includes ameliorating at least one physical parameter, which may be indiscernible by the subject. In yet another embodiment, “treating” or “treatment” includes modulating the disease or disorder, either physically (e.g., stabilization of a discernible symptom) or physiologically (e.g., stabilization of a physical parameter) or both. In yet another embodiment, “treating” or “treatment” includes delaying the onset of the disease or disorder.

“Therapeutically effective amount” refers to an amount of an agent or composition that, when administered to a subject for treating a disease, is sufficient to effect such treatment for the disease. A “therapeutically effective amount” can vary depending on, inter alia, the agent, the disease and its severity, and the age, weight, etc., of the subject to be treated.

As used herein, the phrase “prophylactically effective amount” refers to the amount of a therapy (e.g., prophylactic agent) which is sufficient to result in the prevention or reduction of the development, recurrence or onset of one or more symptoms associated with a disorder or to enhance or improve the prophylactic effect(s) of another therapy (e.g., another prophylactic agent).

Compositions

Provided herein are compositions useful for the treatment or prevention of C. difficile-associated diseases such as C. difficile-associated diarrhea in a subject in need thereof. The compositions can be formed as described herein and administered for the treatment or prevention of C. difficile-associated disease. The compositions are formulated for administration to a subject, in particular embodiments to a human. The compositions can be of any form deemed useful by the practitioner of skill. In certain embodiments, the compositions are medical foods, or a component thereof. In certain embodiments, the compositions are nutritional supplements, nutraceuticals, or components thereof. In certain embodiments, the compositions comprise smectite. In certain embodiments, the smectite is di-tri-octahedral smectite.

The term medical food is defined in section 5(b) of the Orphan Drug Act (21 U.S.C. 360ee (b) (3)) as a food which is formulated to be consumed or administered enterally under the supervision of a physician and which is intended for the specific dietary management of a disease or condition for which distinctive nutritional requirements, based on recognized scientific principles, are established by medical evaluation. Medical foods are distinguished from the broader category of foods for special dietary use and from foods that make health claims by the requirement that medical foods be intended to meet distinctive nutritional requirements of a disease or condition, used under medical supervision and intended for the specific dietary management of a disease or condition. The term “medical foods” does not pertain to all foods fed to sick patients. Medical foods are foods that are specially formulated and processed (as opposed to a naturally occurring foodstuff used in a natural state) for the patient who is seriously ill or who requires the product as a major treatment modality. In general, to be considered a medical food, a product meets the following criteria: the product is a food for oral or tube feeding; the product is labeled for the dietary management of a specific medical disorder, disease, or condition for which there are distinctive nutritional requirements; and the product is intended to be used under medical supervision. See Food Labeling; Reference Daily Intakes and Daily Reference Values; Mandatory Status of Nutrition Labeling and Nutrition Content Revision proposed rule (56 FR 60366 at 60377, Nov. 27, 1991).

In certain embodiments, provided herein is a composition comprising smectite. The smectite can be present in any amount deemed suitable to the practitioner of skill in the art. In certain embodiments, the composition comprises an amount of smectite effective to treat or prevent C. difficile associated disease in a subject in need thereof. In certain embodiments, the composition comprises an amount of smectite effective to treat or prevent a C. difficile-associated disease in a subject in need thereof. In certain embodiments, the composition comprises an amount of smectite effective to treat or prevent C. difficile-associated diarrhea in a subject in need thereof. The composition can be a medical food, a nutritional supplement, or a nutraceutical, or a component thereof.

The composition can be in any form deemed suitable by those of skill in the art. In certain embodiments, the composition can be a liquid, a suspension, a liquid for oral administration, a suspension for oral administration, a liquid for tube feeding or a suspension for tube feeding. The composition can also be a solid, for instance a solid food for oral consumption. The solid can be in any form deemed suitable to those of skill. In certain embodiments, the solid is in the form of a powder or flakes. They can be used, for example, for dissolution into a liquid or for suspension, or for sprinkling. In certain embodiments, the solid is in the form of a tablet or capsule. In certain embodiments, the solid is in the form of a food that can be eaten, such as a bar, gel, block, etc.

In some embodiments, the composition comprises 1 mg to 100 g smectite. In certain embodiments, the composition comprises 1 mg to 50 g smectite. In certain embodiments, the composition comprises 1 mg to 25 g smectite. In certain embodiments, the composition comprises 1 mg to 20 g smectite. In certain embodiments, the composition comprises 1 mg to 18 g smectite. In certain embodiments, the composition comprises 100 mg to 18 g smectite. In certain embodiments, the composition comprises 500 mg to 18 g smectite. In certain embodiments, the composition comprises 800 mg to 18 g smectite. In certain embodiments, the composition comprises 1600 mg to 18 g smectite. The composition can be a medical food, a nutritional supplement, or a nutraceutical, or a component thereof.

The smectite can be any smectite deemed suitable to those of skill in the art. In certain embodiments, the smectite is di-octahedral smectite. In certain embodiments, the smectite is di-tri-octahedral smectite. In some embodiments, the composition comprises 1 mg to 100 g di-tri-octahedral smectite. In certain embodiments, the composition comprises 1 mg to 50 g di-tri-octahedral smectite. In certain embodiments, the composition comprises 1 mg to 25 g di-tri-octahedral smectite. In certain embodiments, the composition comprises 1 mg to 20 g di-tri-octahedral smectite. In certain embodiments, the composition comprises 1 mg to 18 g di-tri-octahedral smectite. In certain embodiments, the composition comprises 100 mg to 18 g di-tri-octahedral smectite. In certain embodiments, the composition comprises 500 mg to 18 g di-tri-octahedral smectite. In certain embodiments, the composition comprises 800 mg to 18 g di-tri-octahedral smectite. In certain embodiments, the composition comprises 1600 mg to 18 g di-tri-octahedral smectite. The composition can be a medical food, a nutritional supplement, or a nutraceutical, or a component thereof.

In certain embodiments, a composition is provided herein which comprises smectite wherein the smectite is the only active component of the composition. In some embodiments, a composition is provided herein which comprises smectite wherein the smectite is the only active component of the composition which is effective for the treatment or prevention of C. difficile-associated disease or C. difficile-associated diarrhea in a subject in need thereof. In some embodiments, a composition is provided herein which comprises smectite wherein the composition does not comprise a second active agent. In some embodiments, a composition is provided herein which comprises smectite and a second agent effective for the treatment of a Clostridium difficile associated disease, or a symptom thereof, in a subject, wherein the smectite and second agent are the only active components of the composition. In some embodiments, a composition is provided herein which comprises smectite and a second agent effective for the treatment of a Clostridium difficile associated disease, or a symptom thereof, in a subject, wherein the composition does not comprise any other active agents.

Of course, in such embodiments the composition can comprise other ingredients for any purpose deemed suitable by practitioners of skill in the art. The other ingredients can be, for example, nutrients, vitamins, fillers, etc. In certain embodiments, the composition can comprise one or more other ingredients effective for treating or preventing another infection, disease or condition, i.e. other than C. difficile-associated disease or C. difficile-associated diarrhea.

In certain embodiments, the composition comprises one or more second agents that are effective for the treatment or prevention of C. difficile-associated disease or C. difficile-associated diarrhea in a subject in need thereof. The second agent can be any second agent known to be effective by the practitioner of skill in the art. In certain embodiments, the second agent is an amino acid, a peptide, a prebiotic, a probiotic, or a combination thereof.

In certain embodiments, the second agent is an amino acid or a peptide. The amino acid or peptide can be any amino acid or peptide effective for the treatment or prevention of C. difficile-associated disease or C. difficile-associated diarrhea in a subject in need thereof. In certain embodiments, the amino acid or peptide is L-glutamine or L-alanyl-L-glutamine. In certain embodiments, the amino acid or peptide is in an amount effective for the treatment or prevention of C. difficile-associated disease or C. difficile-associated diarrhea in a subject in need thereof. While not intending to be bound by any particular theory of operation, certain embodiments are based on the observation that L-glutamine is capable of providing energy to enterocytes (intestinal cells) which can improve gut integrity after stress or injury, repair damaged gut tissue, increase resistance to infectious disease, reduce bacterial translocation, and provide a nitrogen source for nucleic acids.

In some embodiments, the composition comprises 0.1 to 60 g L-glutamine or L-alanyl-L-glutamine. In certain embodiments, the composition comprises 0.1 to 50 g L-glutamine or L-alanyl-L-glutamine. In certain embodiments, the composition comprises 0.1 to 40 g L-glutamine or L-alanyl-L-glutamine. In certain embodiments, the composition comprises 0.1 to 30 g L-glutamine or L-alanyl-L-glutamine. In certain embodiments, the composition comprises 0.1 to 25 g L-glutamine or L-alanyl-L-glutamine. In certain embodiments, the composition comprises 0.1 to 20 g L-glutamine or L-alanyl-L-glutamine. In certain embodiments, the composition comprises 0.1 to 15 g L-glutamine or L-alanyl-L-glutamine. The composition can be a medical food, a nutritional supplement, or a nutraceutical, or a component thereof.

In some embodiments, the composition comprises 1 mg to 100 g smectite and 0.1 to 60 g L-glutamine. In some embodiments, the composition comprises 1 mg to 18 g smectite and 0.1 to 60 g L-glutamine. In certain embodiments, the composition comprises 100 mg to 18 g smectite and 0.1 to 60 g L-glutamine. In certain embodiments, the composition comprises 800 mg to 18 g smectite and 0.1 to 60 g L-glutamine. In certain embodiments, the composition comprises 1600 mg to 18 g smectite and 0.1 to 60 g L-glutamine. The composition can be a medical food, a nutritional supplement, or a nutraceutical, or a component thereof.

In some embodiments, the composition comprises 1 mg to 100 g di-tri-octahedral smectite and 0.1 to 60 g L-glutamine. In some embodiments, the composition comprises 1 mg to 18 g di-tri-octahedral smectite and 0.1 to 60 g L-glutamine. In certain embodiments, the composition comprises 100 mg to 18 g di-tri-octahedral smectite and 0.1 to 60 g L-glutamine. In certain embodiments, the composition comprises 800 mg to 18 g di-tri-octahedral smectite and 0.1 to 60 g L-glutamine. In certain embodiments, the composition comprises 1600 mg to 18 g di-tri-octahedral smectite and 0.1 to 60 g L-glutamine. The composition can be a medical food, a nutritional supplement, or a nutraceutical, or a component thereof.

In some embodiments, the composition comprises 1 mg to 8 g smectite and 0.1 to 60 g L-alanyl-L-glutamine. In some embodiments, the composition comprises 100 mg to 8 g smectite and 0.1 to 60 g L-alanyl-L-glutamine. In certain embodiments, the composition comprises 800 mg to 18 g smectite and 0.1 to 60 g L-alanyl-L-glutamine. In certain embodiments, the composition comprises 1600 mg to 18 g smectite and 0.1 to 60 g L-alanyl-L-glutamine. The composition can be a medical food, a nutritional supplement, or a nutraceutical, or a component thereof.

In some embodiments, the composition comprises 1 mg to 8 g di-tri-octahedral smectite and 0.1 to 60 g L-alanyl-L-glutamine. In some embodiments, the composition comprises 100 mg to 8 g di-tri-octahedral smectite and 0.1 to 60 g L-alanyl-L-glutamine. In certain embodiments, the composition comprises 800 mg to 18 g di-tri-octahedral smectite and 0.1 to 60 g L-alanyl-L-glutamine. In certain embodiments, the composition comprises 1600 mg to 18 g di-tri-octahedral smectite and 0.1 to 60 g L-alanyl-L-glutamine. The composition can be a medical food, a nutritional supplement, or a nutraceutical, or a component thereof.

In certain embodiments, the second agent is a prebiotic. The prebiotic can be any prebiotic deemed effective by a practitioner of skill in the art for the treatment or prevention of C. difficile-associated disease or C. difficile-associated diarrhea in a subject in need thereof. In certain embodiments, the prebiotic is in an amount deemed effective by a practitioner of skill in the art for the treatment or prevention of C. difficile-associated disease or C. difficile-associated diarrhea in a subject in need thereof. While not intending to be bound by any particular theory of operation, certain embodiments are based on the observation that prebiotics can indirectly improve the normal flora of the gut, thereby reducing the risk of colonization by pathogenic bacteria such as C. difficile.

In certain embodiments, the prebiotic is selected from the group consisting of fructooligosaccharides, oligo fructosaccharides, inulin, chicory, xylooligosaccharides, mannan oligosaccharides, lactosucrose, galactooligosaccharides, lactosucrose, pyrodextrin, soy oligosaccharides, transgalactooligosaccharides, isomaltooligosaccharide, lactilol, lactulose, polydextrose, resistant starch, arabinogalactan, and combinations thereof. In particular embodiments, the prebiotic is mannan oligosaccharides (MOS).

In some embodiments, the composition comprises 100 mg to 40 g prebiotic. In certain embodiments, the composition comprises 100 mg to 30 g prebiotic. In certain embodiments, the composition comprises 100 mg to 25 g prebiotic. The composition can be a medical food, a nutritional supplement, or a nutraceutical, or a component thereof.

In some embodiments, the composition comprises 1 mg to 100 g smectite and 1 mg to 40 g prebiotic. In some embodiments, the composition comprises 1 mg to 18 g smectite and 1 mg to 40 g prebiotic. In some embodiments, the composition comprises 100 mg to 18 g smectite and 1 mg to 40 g prebiotic. In some embodiments, the composition comprises 1 mg to 18 g smectite and 100 mg to 40 g prebiotic. In some embodiments, the composition comprises 100 mg to 18 g smectite and 100 mg to 40 g prebiotic. In certain embodiments, the composition comprises 800 mg to 18 g di smectite and 100 mg to 40 g prebiotic. In certain embodiments, the composition comprises 1600 mg to 18 g smectite and 100 mg to 40 g prebiotic. The composition can be a medical food, a nutritional supplement, or a nutraceutical, or a component thereof.

In some embodiments, the composition comprises 1 mg to 100 g di-tri-octahedral smectite and 1 mg to 40 g prebiotic. In some embodiments, the composition comprises 1 mg to 18 g di-tri-octahedral smectite and 1 mg to 40 g prebiotic. In some embodiments, the composition comprises 100 mg to 18 g di-tri-octahedral smectite and 1 mg to 40 g prebiotic. In some embodiments, the composition comprises 1 mg to 18 g di-tri-octahedral smectite and 100 mg to 40 g prebiotic. In some embodiments, the composition comprises 100 mg to 18 g di-tri-octahedral smectite and 100 mg to 40 g prebiotic. In certain embodiments, the composition comprises 800 mg to 18 g di-tri-octahedral smectite and 100 mg to 40 g prebiotic. In certain embodiments, the composition comprises 1600 mg to 18 g di-tri-octahedral smectite and 100 mg to 40 g prebiotic. The composition can be a medical food, a nutritional supplement, or a nutraceutical, or a component thereof.

In certain embodiments, the second agent is a probiotic. The probiotic can be any probiotic deemed effective by a practitioner of skill in the art for the treatment or prevention of C. difficile-associated disease or C. difficile-associated diarrhea in a subject in need thereof. In certain embodiments, the probiotic is in an amount deemed effective by a practitioner of skill in the art for the treatment or prevention of C. difficile-associated disease or C. difficile-associated diarrhea in a subject in need thereof. The composition can be a medical food, a nutritional supplement, or a nutraceutical, or a component thereof. While not intending to be bound by any particular theory of operation, certain embodiments are based on the observation that probiotics can improve the normal flora of the gut, thereby reducing the risk of colonization by pathogenic bacteria such as C. difficile.

In certain embodiments, the probiotic is selected from the group consisting of the yeast Saccharomyces boulardii; and the bacteria Bifidobacterium, Lactobacillus, and propionibacteria such as: Bifidobacterium animalis subsp. lactis; Bifidobacterium bifidum; Bifidobacterium breve; Bifidobacterium infantis; Bifidobacterium longum; Lactobacillus acidophilus; Lactobacillus casei; Lactobacillus plantarum; Lactobacillus reuteri; Lactobacillus rhamnosus; and combinations thereof. Particularly useful probiotics include Saccharomyces, Bifidobacteria and Lactobacillus. In certain embodiments, the probiotic is a Bacillus bacterium.

In some embodiments, the composition comprises 100 million to 500 billion CFU probiotic. In certain embodiments, the composition comprises 100 million to 250 billion CFU probiotic. In certain embodiments, the composition comprises 100 million to 100 billion CFU probiotic. The composition can be a medical food, a nutritional supplement, or a nutraceutical, or a component thereof.

In some embodiments, the composition comprises 1 mg to 100 g smectite and 100 million to 500 billion CFU probiotic. In some embodiments, the composition comprises 1 mg to 18 g smectite and 100 million to 500 billion CFU probiotic. In some embodiments, the composition comprises 100 mg to 18 g and 100 million to 500 billion CFU probiotic. In certain embodiments, the composition comprises 800 mg to 18 g smectite and 100 million to 500 billion CFU probiotic. In certain embodiments, the composition comprises 1600 mg to 18 g smectite and 100 million to 500 billion CFU probiotic. The composition can be a medical food, a nutritional supplement, or a nutraceutical, or a component thereof.

In some embodiments, the composition comprises 1 mg to 100 g di-tri-octahedral smectite and 100 million to 500 billion CFU probiotic. In some embodiments, the composition comprises 1 mg to 18 g di-tri-octahedral smectite and 100 million to 500 billion CFU probiotic. In some embodiments, the composition comprises 100 mg to 18 g di-tri-octahedral smectite and 100 million to 500 billion CFU probiotic. In certain embodiments, the composition comprises 800 mg to 18 g di-tri-octahedral smectite and 100 million to 500 billion CFU probiotic. In certain embodiments, the composition comprises 1600 mg to 18 g di-tri-octahedral smectite and 100 million to 500 billion CFU probiotic. The composition can be a medical food, a nutritional supplement, or a nutraceutical, or a component thereof.

In certain embodiments, the composition comprises one or more second agents from one of the above classes. In certain embodiments, the composition comprises second agents from two of the above classes. In certain embodiments, the composition comprises no active ingredients other than the smectite and the one or more second agents. In certain embodiments, the composition comprises second agents from three or more of the above classes. The composition can be a medical food, a nutritional supplement, or a nutraceutical, or a component thereof.

In certain embodiments, the smectite and the one or more second agents are in amounts that are synergistic. In such embodiments, the composition provides efficacy that is more than an additive combination of the smectite and the one or more second agents. The efficacy can be measured in any aspect or symptom(s) of C. difficile associated disease used by those of skill in the art, such as a reduction in C. difficile associated weight loss, diarrhea (frequency and/or volume), abdominal cramping, fever, nausea, amount of blood or pus in the stool, dehydration, loss of appetite, kidney failure, electrolyte imbalance, and increased white blood cell count.

In certain embodiments, the second agent can be formulated or packaged with the active agent provided herein. Of course, the second agent will only be formulated with the active agent provided herein when, according to the judgment of those of skill in the art, such co-formulation should not interfere with the activity of either agent or the method of administration. In certain embodiments, the active agent and the second agent are formulated separately. They can be packaged together, or packaged separately, for the convenience of the practitioner of skill in the art.

In certain embodiments, any of the above compositions can further comprise one or more pharmaceutical agents. The pharmaceutical agent can be any agent deemed useful by a practitioner of skill. In particular embodiments, the pharmaceutical agent is effective for the treatment or prevention of C. difficile infection, C. difficile-associated disease or C. difficile-associated diarrhea, or any symptom thereof. Examples of useful pharmaceutical agents include antibiotics such as metronidazole and vancomycin. In other embodiments, the pharmaceutical agent is effective for the treatment or prevention of another disease or condition for the subject in need thereof.

Methods

Also provided herein are methods for the treatment and/or prophylaxis of a subject infected with C. difficile that includes the administration of an effective amount of a composition as described herein.

The compositions as described herein can be for use in therapy or prophylaxis, for example in the treatment of a bacterial infection or disease. In certain embodiments, the compositions can be used in the treatment or prevention of C. difficile associated disease in a subject in need thereof. In certain embodiments, provided herein are methods of treating or preventing a bacterial disease, for example C. difficile associated disease or C. difficile associated diarrhea, comprising the step of administering an effective amount of a composition described herein to a subject in need thereof. In certain embodiments, provided herein are methods of treating or preventing a bacterial disease, for example C. difficile associated disease or C. difficile associated diarrhea, comprising the step of contacting a bacterial toxin with a composition described herein.

In certain embodiments, provided herein are methods for the specific dietary management of a C. difficile associated disease, or a symptom thereof, comprising the step of administering to a human subject in need thereof a composition provided herein. In certain embodiments, the subject has limited or impaired capacity to ingest, digest, absorb, or metabolize ordinary foodstuffs. In certain embodiments, the composition provided herein comprises a sufficient amount of smectite for the specific dietary management of a Clostridium difficile associated disease, or a symptom thereof, in the subject in need thereof. In certain embodiments, the composition provided herein further comprises a second agent. In certain embodiments, the second agent is effective for the specific dietary management of a Clostridium difficile associated disease, or a symptom thereof, in the subject. In certain embodiments, the composition provided herein comprises a synergistic amount of the second agent. In certain embodiments, the second agent is selected from the group consisting an amino acid, a peptide, a prebiotic, a probiotic, and combinations thereof. In certain embodiments, the second agent is effective for the specific dietary management of a Clostridium difficile associated disease, or a symptom thereof, in the subject in need thereof. In certain embodiments, the second agent is selected from the group consisting of L-glutamine, L-alanyl L-glutamine, manna oligosaccharides, Saccharomyces boulardii, Lactobacillus, Bifidobacteria, and combinations thereof. In certain embodiments, the second agent is a Bacillus bacterium. In certain embodiments, the subject requires the composition provided herein as a major treatment modality. In certain embodiments, the administering is under medical supervision. In certain embodiments, the smectite is di-tri-octahedral smectite. In certain embodiments, the composition is a medical food, nutritional supplement or nutraceutical.

In certain embodiments, provided herein are methods for the specific dietary management of a C. difficile associated disease, or a symptom thereof, comprising the step of administering to a human subject in need thereof a medical food comprising 1 mg to 100 g smectite. In certain embodiments, the subject has limited or impaired capacity to ingest, digest, absorb, or metabolize ordinary foodstuffs. In certain embodiments, the medical food comprises a sufficient amount of smectite for the specific dietary management of a Clostridium difficile associated disease, or a symptom thereof, in the subject in need thereof. In certain embodiments, the medical food further comprises a second agent. In certain embodiments, the second agent is effective for the specific dietary management of a Clostridium difficile associated disease, or a symptom thereof, in the subject. In certain embodiments, the medical food comprises a synergistic amount of the second agent. In certain embodiments, the second agent is selected from the group consisting an amino acid, a peptide, a prebiotic, a probiotic, and combinations thereof. In certain embodiments, the second agent is selected from the group consisting of L-glutamine, L-alanyl L-glutamine, manna oligosaccharides, Saccharomyces boulardii, Lactobacillus, Bifidobacteria, and combinations thereof. In certain embodiments, the second agent is a Bacillus bacterium. In certain embodiments, the subject requires the medical food as a major treatment modality. In certain embodiments, the administering is under medical supervision. In certain embodiments, the smectite is di-tri-octahedral smectite.

In certain embodiments, provided herein are methods for the specific dietary management of a C. difficile associated disease, or a symptom thereof, comprising the step of administering to a human subject in need thereof a nutritional supplement comprising 1 mg to 100 g smectite. In certain embodiments, the subject has limited or impaired capacity to ingest, digest, absorb, or metabolize ordinary foodstuffs. In certain embodiments, the nutritional supplement comprises a sufficient amount of smectite for the specific dietary management of a Clostridium difficile associated disease, or a symptom thereof, in the subject in need thereof. In certain embodiments, the nutritional supplement further comprises a second agent. In certain embodiments, the second agent is effective for the specific dietary management of a Clostridium difficile associated disease, or a symptom thereof, in the subject. In certain embodiments, the nutritional supplement comprises a synergistic amount of the second agent. In certain embodiments, the second agent is selected from the group consisting an amino acid, a peptide, a prebiotic, a probiotic, and combinations thereof. In certain embodiments, the second agent is selected from the group consisting of L-glutamine, L-alanyl L-glutamine, manna oligosaccharides, Saccharomyces boulardii, Lactobacillus, Bifidobacteria, and combinations thereof. In certain embodiments, the second agent is a Bacillus bacterium. In certain embodiments, the subject requires the nutritional supplement as a major treatment modality. In certain embodiments, the administering is under medical supervision. In certain embodiments, the smectite is di-tri-octahedral smectite.

In certain embodiments, provided herein are methods for the specific dietary management of a C. difficile associated disease, or a symptom thereof, comprising the step of administering to a human subject in need thereof a nutraceutical comprising 1 mg to 100 g smectite. In certain embodiments, the subject has limited or impaired capacity to ingest, digest, absorb, or metabolize ordinary foodstuffs. In certain embodiments, the nutraceutical comprises a sufficient amount of smectite for the specific dietary management of a Clostridium difficile associated disease, or a symptom thereof, in the subject in need thereof. In certain embodiments, the nutraceutical further comprises a second agent. In certain embodiments, the second agent is effective for the specific dietary management of a Clostridium difficile associated disease, or a symptom thereof, in the subject. In certain embodiments, the nutraceutical comprises a synergistic amount of the second agent. In certain embodiments, the second agent is selected from the group consisting an amino acid, a peptide, a prebiotic, a probiotic, and combinations thereof. In certain embodiments, the second agent is selected from the group consisting of L-glutamine, L-alanyl L-glutamine, manna oligosaccharides, Saccharomyces boulardii, Lactobacillus, Bifidobacteria, and combinations thereof. In certain embodiments, the second agent is a Bacillus bacterium. In certain embodiments, the subject requires the nutraceutical as a major treatment modality. In certain embodiments, the administering is under medical supervision. In certain embodiments, the smectite is di-tri-octahedral smectite.

In certain embodiments, provided herein are methods for the specific dietary management of a C. difficile associated disease, or a symptom thereof, comprising the step of administering to a human subject in need thereof a composition which comprises smectite wherein the smectite is the only active component of the composition. In some embodiments, provided herein are methods for the specific dietary management of a C. difficile associated disease, or a symptom thereof, comprising the step of administering to a human subject in need thereof a composition which comprises smectite wherein the smectite is the only active component of the composition which is effective for the treatment or prevention of a C. difficile-associated disease or C. difficile-associated diarrhea in the subject in need thereof. In some embodiments, provided herein are methods for the specific dietary management of a C. difficile associated disease, or a symptom thereof, comprising the step of administering to a human subject in need thereof a composition which comprises smectite wherein the composition does not comprise a second active agent. In some embodiments, provided herein are methods for the specific dietary management of a C. difficile associated disease, or a symptom thereof; comprising the step of administering to a human subject in need thereof a composition which comprises smectite and a second agent effective for the treatment of a Clostridium difficile associated disease, or a symptom thereof, in the subject, wherein the smectite and second agent are the only active components of the composition. In some embodiments, provided herein are methods for the specific dietary management of a C. difficile associated disease, or a symptom thereof, comprising the step of administering to a human subject in need thereof a composition which comprises smectite and a second agent effective for the treatment of a Clostridium difficile associated disease, or a symptom thereof, in the subject, wherein the composition does not comprise any other active agents.

In certain embodiments, any of the above compositions can be administered in combination with any other therapy for C. difficile infection, C. difficile-associated disease or C. difficile-associated diarrhea, or any symptom thereof. Examples include pharmaceutical agents, dietary change, bed rest, and intravenous fluid, surgery, and/or fecal transplant. Examples of useful pharmaceutical agents include antibiotics such as metronidazole and vancomycin. In other embodiments, the therapy is effective for the treatment or prevention of another disease or condition for the subject in need thereof.

In certain embodiments, the subject in need has C. difficile infection, C. difficile-associated disease or C. difficile-associated diarrhea, or any symptom thereof. In certain embodiments, the subject in need is at risk for C. difficile infection, C. difficile-associated disease or C. difficile-associated diarrhea, or any symptom thereof. In certain embodiments, the subject is undergoing antibiotic therapy or expected to undergo antibiotic therapy. In certain embodiments, the subject is in surgery, pre-surgery or post-surgery procedures.

Preparation of Compositions

The compositions provided herein can be prepared or obtained by any method apparent to those of skill in the art. Exemplary methods of preparation are described in the examples below. In certain embodiments, the components of the compositions are obtained from commercial sources. They can be mixed or combined according to standard practice. In preferred embodiments, the compositions are prepared according to current Good Manufacturing Practices. The compositions can be packaged and delivered as described herein.

Formulations and Methods of Administration

The active agents are formulated into compositions using methods available in the art and those disclosed herein.

The methods provided herein encompass administering compositions containing at least one agent as described herein, either alone or in combination with one or more compatible and pharmaceutically acceptable carriers, such as diluents or adjuvants.

In clinical practice the compositions provided herein may be administered by any conventional route, in particular orally or by feeding tube.

Use may be made, as solid compositions for oral administration, of tablets, pills, hard gelatin capsules, powders or granules. In these compositions, the active product may be mixed with one or more inert diluents or adjuvants, such as sucrose, lactose or starch.

These compositions can comprise substances other than diluents, for example a lubricant, such as magnesium stearate, or a coating intended for controlled release.

Use may be made, as liquid compositions for oral administration, of solutions which are pharmaceutically acceptable, suspensions, emulsions, syrups and elixirs containing inert diluents, such as water or liquid paraffin. These compositions can also comprise substances other than diluents, for example wetting, sweetening or flavoring products.

The compositions can be emulsions or sterile solutions. Use may be made, as solvent or vehicle, of propylene glycol, a polyethylene glycol, vegetable oils, in particular olive oil, or injectable organic esters, for example ethyl oleate. These compositions can also contain adjuvants, in particular wetting, isotonizing, emulsifying, dispersing and stabilizing agents. Where appropriate (e.g., in compositions that do not comprise probiotics), sterilization can be carried out in several ways, for example using a bacteriological filter, by radiation or by heating. They can also be prepared in the form of sterile solid compositions which can be dissolved at the time of use in sterile water or any other injectable sterile medium.

In certain embodiments, a composition provided herein is a single unit dosage form. Single unit dosage forms provided herein comprise a prophylactically or therapeutically effective amount of one or more active agents and typically one or more pharmaceutically acceptable carriers or excipients. In a specific embodiment and in this context, the term “pharmaceutically acceptable” means approved by a regulatory agency of the Federal or a state government or listed in the U.S. Pharmacopeia or other generally recognized pharmacopeia for use in animals, and more particularly in humans. The term “carrier” includes a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water can be used as a carrier when the composition is administered intravenously. Saline solutions and aqueous dextrose and glycerol solutions can also be employed as liquid carriers, particularly for injectable solutions. Examples of suitable pharmaceutical carriers are described in “Remington's Pharmaceutical Sciences” by E. W. Martin.

Typical compositions and dosage forms comprise one or more excipients. Suitable excipients are well-known to those skilled in the art of pharmacy, and non-limiting examples of suitable excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. Whether a particular excipient is suitable for incorporation into a composition or dosage form depends on a variety of factors well known in the art including, but not limited to, the way in which the dosage form will be administered to a subject and the specific active ingredients in the dosage form. The composition or single unit dosage form, if desired, can also contain minor amounts of wetting or emulsifying agents, or pH buffering agents.

Lactose free compositions provided herein can comprise excipients that are well known in the art and are listed, for example, in the U.S. Pharmocopia (USP) SP (XXI)/NF (XVI). In general, lactose free compositions comprise an active ingredient, a binder/filler, and a lubricant in pharmaceutically compatible and pharmaceutically acceptable amounts. Exemplary lactose free dosage forms comprise an active ingredient, microcrystalline cellulose, pre gelatinized starch, and magnesium stearate.

Further provided are compositions and dosage forms that comprise one or more compounds that reduce the rate by which an active agent will decompose. Such compounds, which are referred to herein as “stabilizers,” include, but are not limited to, antioxidants such as ascorbic acid, pH buffers, or salt buffers.

The compositions and single unit dosage forms can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Such compositions and dosage forms will contain a prophylactically or therapeutically effective amount of a prophylactic or therapeutic agent, in certain embodiments, in purified form, together with a suitable amount of carrier so as to provide the form for proper administration to the subject. The formulation should suit the mode of administration. In a certain embodiment, the compositions or single unit dosage forms are sterile and in suitable form for administration to a subject, for example, an animal subject, such as a mammalian subject, for example, a human subject.

A composition is formulated to be compatible with its intended route of administration. Examples of routes of administration include, but are not limited to, oral and by feeding tube. In a specific embodiment, the composition is formulated in accordance with routine procedures adapted for oral administration to human beings.

Examples of dosage forms include, but are not limited to: tablets; caplets; capsules, such as soft elastic gelatin capsules; cachets; troches; lozenges; gels; liquid dosage forms suitable for oral administration to a subject, including suspensions (e.g., aqueous or nonaqueous liquid suspensions, oil in water emulsions, or a water in oil liquid emulsions), solutions, and elixirs; and powders that can be reconstituted to provide liquid dosage forms suitable for oral administration to a subject.

Generally, the ingredients of compositions are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline.

Typical dosage forms comprise an agent provided herein, within the range of from about 0.1 mg to about 1000 g per day, given as a single once-a-day dose in the morning or as divided doses throughout the day. In some embodiments, particular dosage forms can have about 0.1, 0.2, 0.3, 0.4, 0.5, 1.0, 2.0, 2.5, 5.0, 10.0, 15.0, 20.0, 25.0, 50.0, 100, 200, 250, 500 or 1000 mg of the active agent. In some embodiments, particular dosage forms can have about 0.1, 0.2, 0.3, 0.4, 0.5, 1.0, 2.0, 2.5, 5.0, 10.0, 15.0, 20.0, 25.0, 50.0, 100, 200, 250, 500 or 1000 g of the active agent.

Oral Dosage Forms

Compositions that are suitable for oral administration can be presented as discrete dosage forms, such as, but are not limited to, powders, tablets (e.g., chewable tablets), caplets, capsules, and liquids (e.g., flavored syrups). Such dosage forms contain predetermined amounts of active ingredients, and may be prepared by methods of pharmacy well known to those skilled in the art. See generally, Remington's Pharmaceutical Sciences, 20th ed., Mack Publishing, Easton Pa. (2000).

In certain embodiments, the oral dosage forms are solid and prepared under anhydrous conditions with anhydrous ingredients, as described in detail in the sections above. However, the scope of the compositions provided herein extends beyond anhydrous, solid oral dosage forms. As such, further forms are described herein.

Typical oral dosage forms are prepared by combining the active agent in an intimate admixture with at least one excipient according to conventional pharmaceutical compounding techniques. Excipients can take a wide variety of forms depending on the form of preparation desired for administration. For example, excipients suitable for use in oral liquid or aerosol dosage forms include, but are not limited to, water, glycols, oils, alcohols, flavoring agents, preservatives, and coloring agents. Examples of excipients suitable for use in solid oral dosage forms (e.g., powders, tablets, capsules, and caplets) include, but are not limited to, starches, sugars, micro crystalline cellulose, diluents, granulating agents, lubricants, binders, and disintegrating agents.

If desired, tablets can be coated by standard aqueous or nonaqueous techniques. Such dosage forms can be prepared by any of the methods of pharmacy. In general, compositions and dosage forms are prepared by uniformly and intimately admixing the active ingredients with liquid carriers, finely divided solid carriers, or both, and then shaping the product into the desired presentation if necessary.

For example, a tablet can be prepared by compression or molding. Compressed tablets can be prepared by compressing in a suitable machine the active ingredients in a free flowing form such as powder or granules, optionally mixed with an excipient. Molded tablets can be made by molding in a suitable machine a mixture of the powdered compound moistened with an inert liquid diluent.

Examples of excipients that can be used in oral dosage forms include, but are not limited to, binders, fillers, disintegrants, and lubricants. Binders suitable for use in compositions and dosage forms include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre gelatinized starch, hydroxypropyl methyl cellulose, (e.g., Nos. 2208, 2906, 2910), microcrystalline cellulose, and mixtures thereof.

Examples of fillers suitable for use in the compositions and dosage forms disclosed herein include, but are not limited to, talc, calcium carbonate (e.g., granules or powder), microcrystalline cellulose, powdered cellulose, dextrates, kaolin, mannitol, silicic acid, sorbitol, starch, pre gelatinized starch, and mixtures thereof. The binder or filler in compositions is typically present in from about 50 to about 99 weight percent of the composition or dosage form.

Suitable forms of microcrystalline cellulose include, but are not limited to, the materials sold as AVICEL PH 101, AVICEL PH 103 AVICEL RC 581, AVICEL PH 105 (available from FMC Corporation, American Viscose Division, Avicel Sales, Marcus Hook, Pa.), and mixtures thereof. A specific binder is a mixture of microcrystalline cellulose and sodium carboxymethyl cellulose sold as AVICEL RC 581. Suitable anhydrous or low moisture excipients or additives include AVICEL PH 103™ and Starch 1500 LM.

Disintegrants are used in the compositions to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms. The amount of disintegrant used varies based upon the type of formulation, and is readily discernible to those of ordinary skill in the art. Typical compositions comprise from about 0.5 to about 15 weight percent of disintegrant, specifically from about 1 to about 5 weight percent of disintegrant.

Disintegrants that can be used in compositions and dosage forms include, but are not limited to, agar, alginic acid, calcium carbonate, microcrystalline cellulose, croscarmellose sodium, crospovidone, polacrilin potassium, sodium starch glycolate, potato or tapioca starch, pre gelatinized starch, other starches, clays, other algins, other celluloses, gums, and mixtures thereof.

Lubricants that can be used in compositions and dosage forms include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, and mixtures thereof. Additional lubricants include, for example, a syloid silica gel (AEROSIL 200, manufactured by W.R. Grace Co. of Baltimore, Md.), a coagulated aerosol of synthetic silica (marketed by Degussa Co. of Plano, Tex.), CAB 0 SIL (a pyrogenic silicon dioxide product sold by Cabot Co. of Boston, Mass.), and mixtures thereof. If used at all, lubricants are typically used in an amount of less than about 1 weight percent of the compositions or dosage forms into which they are incorporated.

Dosage and Unit Dosage Forms

In human therapeutics, a doctor will determine the posology which he considers most appropriate according to a preventive or curative treatment and according to the age, weight, stage of the disease and other factors specific to the subject to be treated. In certain embodiments, doses are from about 1 mg to about 1000 g per day for an adult, or from about 5 to about 250 g per day or from about 10 to 50 g per day for an adult. In certain embodiments, doses are from about 5 to about 400 g per day or 25 to 200 g per day per adult. In certain embodiments, dose rates of from about 50 to about 500 g per day are also contemplated.

In further aspects, provided are methods for the treatment or prevention of C. difficile-associated disease or C. difficile-associated diarrhea in a subject in need thereof in a subject by administering, to a subject in need thereof, an effective amount of an agent provided herein. The amount of the agent or composition which will be effective in the prevention or treatment of a disorder or one or more symptoms thereof will vary with the nature and severity of the disease or condition, and the route by which the active ingredient is administered. The frequency and dosage will also vary according to factors specific for each subject depending on the specific therapy (e.g., therapeutic or prophylactic agents) administered, the severity of the disorder, disease, or condition, the route of administration, as well as age, body, weight, response, and the past medical history of the subject. Effective doses may be extrapolated from dose-response curves derived from in vitro or animal model test systems.

In certain embodiments, the recommended daily dose range of a composition provided herein for the conditions described herein lie within the range of from about 1 mg to about 1000 g per day, given as a single once-a-day dose or as divided doses throughout a day. In certain embodiments, the daily dose is administered twice daily in equally divided doses. In certain embodiments, a daily dose range should be from about 10 g to about 200 g per day, in other embodiments, between about 10 g and about 150 g per day, in further embodiments, between about 25 and about 100 g per day. It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with subject response.

In certain embodiment, the dosage of the composition provided herein, based on weight of the active agent, administered to prevent, treat, manage, or ameliorate a disorder, or one or more symptoms thereof in a subject is 0.1 g/kg, 1 g/kg, 2 g/kg, 3 g/kg, 4 g/kg, 5 g/kg, 6 g/kg, 10 g/kg, or 15 g/kg or more of a subject's body weight. In another embodiment, the dosage of the composition or a composition provided herein administered to prevent, treat, manage, or ameliorate a disorder, or one or more symptoms thereof in a subject is a unit dose of 0.1 g to 200 g, 0.1 g to 100 g, 0.1 g to 50 g, 0.1 g to 25 g, 0.1 g to 20 g, 0.1 g to 15 g, 0.1 g to 10 g, 0.1 g to 7.5 g, 0.1 g to 5 g, 0.1 to 2.5 g, 0.25 g to 20 g, 0.25 to 15 g, 0.25 to 12 g, 0.25 to 10 g, 0.25 g to 7.5 g, 0.25 g to 5 g, 0.5 g to 2.5 g, 1 g to 20 g, 1 g to 15 g, 1 g to 12 g, 1 g to 10 g, 1 g to 7.5 g, 1 g to 5 g, or 1 g to 2.5 g.

In certain embodiments, administration of the same composition may be repeated and the administrations may be separated by at least 1 day, 2 days, 3 days, 5 days, 10 days, 15 days, 30 days, 45 days, 2 months, 75 days, 3 months, or 6 months. In other embodiments, administration of the same prophylactic or therapeutic agent may be repeated and the administration may be separated by at least at least 1 day, 2 days, 3 days, 5 days, 10 days, 15 days, 30 days, 45 days, 2 months, 75 days, 3 months, or 6 months.

In certain aspects, provided herein are unit dosages comprising an agent in a form suitable for administration. Such forms are described in detail above. In certain embodiments, the unit dosage comprises 1 to 1000 g, 5 to 250 g or 10 to 50 g active ingredient. In particular embodiments, the unit dosages comprise about 1, 5, 10, 25, 50, 100, 125, 250, 500 or 1000 g active ingredient. Such unit dosages can be prepared according to techniques familiar to those of skill in the art.

Package Systems and Delivery Systems

Also provided are package systems for the compositions and their use. The package systems can include a composition provided herein and instructions providing information to a health care provider regarding usage. Instructions may be provided in printed form or in the form of an electronic medium such as a flash memory, CD, or DVD, or in the form of a website address where such instructions may be obtained.

In some embodiments, suitable packaging is provided. As used herein, “packaging” includes a solid matrix or material customarily used in a system and capable of holding within fixed limits a composition provided herein suitable for administration to a subject. Such materials include glass and plastic (e.g., polyethylene, polypropylene, and polycarbonate) bottles, vials, paper, plastic, and plastic-foil laminated envelopes and the like. If e-beam sterilization techniques are employed, the packaging should have sufficiently low density to permit sterilization of the contents.

In certain embodiments, provided herein are packages comprising one or more unit dosages of a composition provided herein. The packages can provide one dose, two doses, three doses, four doses, five doses, six doses, seven doses, eight doses, nine doses, ten doses, eleven doses, twelve doses, thirteen doses, fourteen doses or more doses of the composition. Conveniently, each dose can be provided in a separate compartment of the package. Each single compartment is configured to dispense the single unit dose without dispensing the other unit doses. In certain embodiments, each unit dose is provided in a blister compartment that can be pierced or broken to dispense a dose. The several compartments of a single package can provide doses for a single day, for several days, for one week, for two weeks or more. Advantageously, the packages can be mailed to a provider or subject to provide access to the unit doses. Each compartment can further provide other compositions for the subject, for example, vitamins, nutrients, nutraceuticals, medications, etc. Further provided herein are packages specifically tailored for a subject in need including in each compartment an effective amount or a unit dose of a composition provided herein and, optionally, one or more additional compositions to be administered with the unit dose.

In certain embodiments, provided herein are delivery systems capable of delivering one or more compositions provided herein. The delivery systems typically comprise one or more compositions and one or more devices for delivering the compositions. The delivery devices can be selected from tubes, syringes, and the like.

### EXAMPLES

As used herein, the symbols and conventions used in these processes, schemes and examples, regardless of whether a particular abbreviation is specifically defined, are consistent with those used in the contemporary scientific literature.

### Examples 1-12

Compositions are prepared with the following ingredients according to good manufacturing practice.

| Exam- | | | | |
|---|---|---|---|---|
| ple | Smectite | Peptide | Prebiotic | Probiotic |
| 1 | X | | | |
| 2 | X | L-glutamine | | |
| 3 | X | L-alanyl-L- | | |
| | | glutamine | | |
| 4 | X | | Mannan | |
| | | | oligosaccharides | |
| 5 | X | | | S. boulardii, |
| | | | | Lactobacillus, |
| | | | | Bifidobacterium |
| | | | | and/or Bacillus |
| 6 | X | L-glutamine | Mannan | |
| | | | oligosaccharides | |
| 7 | X | L-glutamine | Mannan | S. boulardii, |
| | | | oligosaccharides | Lactobacillus, |
| | | | | Bifidobacterium |
| | | | | and/or Bacillus |
| 8 | X | L-alanyl-L- | Mannan | |
| | | glutamine | oligosaccharides | |
| 9 | X | L-alanyl-L- | Mannan | S. boulardii, |
| | | glutamine | oligosaccharides | Lactobacillus, |
| | | | | Bifidobacterium |
| | | | | and/or Bacillus |
| 10 | X | L-glutamine | | S. boulardii, |
| | | | | Lactobacillus, |
| | | | | Bifidobacterium |
| | | | | and/or Bacillus |
| 11 | X | L-alanyl-L- | | S. boulardii, |
| | | glutamine | | Lactobacillus, |
| | | | | Bifidobacterium |
| | | | | and/or Bacillus |
| 12 | X | | Mannan | S. boulardii, |
| | | | oligosaccharides | Lactobacillus, |
| | | | | Bifidobacterium |
| | | | | and/or Bacillus |

### Example 13

Rats bearing gastrointestinal C. difficile infections are fed the compositions of Examples 1-13. Symptoms of C. difficile associated disease are measured over time. The results for the compositions of Examples 1-13 are compared.

All publications, patents, and patent applications cited in this specification are herein incorporated by reference as if each individual publication, patent or patent application were specifically and individually indicated to be incorporated by reference. While the claimed subject matter has been described in terms of various embodiments, the skilled artisan will appreciate that various modifications, substitutions, omissions, and changes may be made without departing from the spirit thereof. Accordingly, it is intended that the scope of the subject matter is limited solely by the scope of the following claims, including equivalents thereof.

What is claimed is:

1. A composition comprising 1 mg to 100 g smectite and at least one further agent that is effective to treat or prevent Clostridium difficile infection, Clostridium difficile associated disease, or a symptom thereof, in a subject in need thereof.

2. The composition of claim 1, wherein the smectite is selected from di-tri-octahedral smectite, bentonite, and combinations thereof.

3. The composition of claim 1 comprising a sufficient amount of smectite to treat or prevent Clostridium difficile associated disease, or a symptom thereof, in a subject in need thereof.

4. The composition of claim 1 comprising a sufficient amount of the at least one further agent to treat or prevent Clostridium difficile associated disease, or a symptom thereof, in a subject in need thereof.

5. The composition of claim 1 that comprises a synergistic amount of the at least one further agent.

6. The composition of claim 1, wherein the at least one further agent is selected from an amino acid, a peptide, a prebiotic, a probiotic, and combinations thereof.

7. The composition of claim 6 wherein the at least one further agent is selected from L-glutamine, L-alanyl L-glutamine, manna oligosaccharides, Saccharomyces boulardii, Lactobacillus, Bifidobacteria, Bacillus, and combinations thereof.

8. The composition of claim 7, wherein the at least one further agent comprises Saccharomyces boulardii and L-glutamine.

9. The composition of claim 8, wherein the smectite is di-tri-octahedral smectite.

10. The composition of claim 1, wherein the composition is a medical food, nutritional supplement or nutraceutical.

11. The composition of claim 1, wherein the at least one further agent comprises a pharmaceutical agent for the treatment or prevention of Clostridium difficile infection, Clostridium difficile associated disease, or a symptom thereof.

12. A method of treating or preventing Clostridium difficile associated disease, or a symptom thereof, comprising the step of administering a composition according to claim 1 to a subject in need thereof.

13. The method of claim 12 wherein at least one further agent is effective for the specific dietary management of a Clostridium difficile associated disease, or a symptom thereof, in the subject.

14. The method of claim 12 wherein the composition comprises a synergistic amount of the at least one further agent.

15. The method of claim 12, wherein the at least one further agent is selected from an amino acid, a peptide, a prebiotic, a probiotic, and combinations thereof.

16. The method of claim 15 wherein the at least one further agent is selected from L-glutamine, L-alanyl L-glutamine, manna oligosaccharides, Saccharomyces boulardii, Lactobacillus, Bifidobacteria, Bacillus and combinations thereof.

17. The method of claim 16, wherein the at least one further agent comprises Saccharomyces boulardii and L-glutamine.

18. The method of claim 17, wherein the smectite is di-tri-octahedral smectite.

19. The method of claim 12, wherein the subject requires the composition as a major treatment modality.

20. The method of claim 12, wherein the administering is under medical supervision.

21. The method of claim 12, wherein the smectite is selected from di-tri-octahedral smectite, bentonite, and combinations thereof.

22. The method of claim 12, wherein the composition is a medical food, nutritional supplement or nutraceutical.

23. The method of claim 12, wherein the at least one further agent comprises a pharmaceutical agent for the treatment or prevention of Clostridium difficile infection, Clostridium difficile associated disease, or a symptom thereof.

24. The method of claim 12, wherein the Clostridium difficile associated disease, or a symptom thereof is selected from diarrhea and weight loss.

25. A method for the specific dietary management of a Clostridium difficile associated disease, or a symptom thereof, comprising the step of administering to a human subject in need thereof a composition of claim 1.

26. The method of claim 25, wherein the subject has limited or impaired capacity to ingest, digest, absorb, or metabolize ordinary foodstuffs.

27. The method of claim 25, wherein the composition comprises a sufficient amount of smectite for the specific dietary management of a Clostridium difficile associated disease, or a symptom thereof, in the subject in need thereof.
